(19) United States
(12) Patent Application Publication
(10) Pub. No.: US 2008/0274208 A1
(43) Pub. Date: Nov. 6, 2008

(54) Water treatment containing DBNPA for use in sanitizing recreational water

(76) Inventors: Michael Joseph Unhoch, Tyrone, GA (US); Sungmee Choi, Cheshire, CT (US); Katherine P. Roberts, Derby, CT (US); Leon Peter O'Malley, Hengoed (GB)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 12/150,592

(22) Filed: Apr. 29, 2008

Related U.S. Application Data
(60) Provisional application No. 60/927,804, filed on May 4, 2007.

(51) Int. Cl. A61K 33/34; A01P 1/00; A01P 15/00; A01P 13/00

(52) U.S. Cl. 424/638

(57) ABSTRACT

The present invention is directed to a composition for reducing the levels of microorganisms in recreational water systems, comprising: (1) a biocidal effective amount of dibromonitrilopropionamide (DBNPA); (2) optionally, a biocidal effective amount of an algaecide selected from the group consisting of didecyldimethylammonium chloride (DDAC), zinc, and copper; and (3) optionally, a compound capable of in situ activation to form an oxidizing agent; wherein the composition is effective for reducing the levels of microorganisms in recreational water systems. The present invention is also directed to a method of controlling the growth of microorganisms in recreational water systems, comprising the step of providing the above composition; and adding the composition to a recreational water system.

### CROSS-REFERENCE TO RELATED APPLICATIONS

This Application claims the benefit of U.S. Provisional Patent Application Ser. No. 60/927,804 filed May 4, 2007.

### BACKGROUND OF THE INVENTION

1. Field of the Invention

The present invention relates to methods and compositions for treatment of recreational water, and more specifically to treatment of pool and spa water using 2,2-dibromo-3-nitrolopropionamide (DBNPA), optionally, an algaecide, and optionally an oxidizer and adjuvant.

2. Description of the Related Art

2,2-dibromo-3-nitrolopropionamide (DBNPA) is known for use as a biocide for cooling tower, pulp and paper and dry cleaning fluids. The prior art is replete with examples of DBNPA compositions, for example U.S. Pat. Nos. 7,208,511; 7,067,063; 7,008,545; 6,796,436; 6,733,654; 6,699,684; 6,478,973; and 6,369,104.

DBNPA is available in various forms, including liquid, granular solid, powder solid, and tablet solid. Each of these forms has advantages and disadvantages, depending on the specific application. A liquid form is convenient for quickly establishing a residual amount of DBNPA in the pool or spa water, as well as remediation of bacterial, algal and fungal problems. Liquid DBNPA can be broadcast over the surface of the water, added to the pool skimmer with the circulation system running, pre-diluted in water, and added to the pool or added by means of an automated dosing system. DBNPA in granular or tablet form offers the advantage of slow and controlled release more effectively delivering a continuous level of biocide by means of skimmer, floater or erosion-type or automated feeders. Granular and tablet forms also offer lower shipping weight, less storage space, minimal spill hazards, and generally safer handling.

One important feature of a successful swimming pool or spa biocide is the stability (e.g., maintenance of a residual level) of the biocide in application. Stability is important to efficacy in application especially when very low concentrations (e.g., 0.2-5 ppm) of active ingredient are needed to control microorganisms in pool and spa water. Five major mechanisms can adversely affect stability and ultimately efficacy in swimming pools and spas. They are pH, sunlight, temperature, hydrolysis, and bather load. Few biocides are impervious to all of these mechanisms. For example, chlorine-release biocides are generally not stable to sunlight and require a stabilizer like cyanuric acid in order to maintain residual levels in water. Bromine-release biocides, on the other hand, cannot currently be stabilized to sunlight and are used only in a small percentage of swimming pools.

There is a need to have available a biocide that is stable in application and efficacious against common swimming pool and spa bacteria, algae and fungi as well as prevent biofilm formation. The present invention is believed to be an answer to that need.

### SUMMARY OF THE INVENTION

In one aspect, the present invention is directed to a composition for reducing the levels of microorganisms in recreational water systems, comprising: (1) a biocidal effective amount of dibromonitrilopropionamide (DBNPA); (2) optionally, a biocidal effective amount of an algaecide selected from the group consisting of didecyldimethylammonium chloride (DDAC), zinc, and copper; and (3) optionally, a compound capable of in situ activation to form an oxidizing agent; wherein the composition is effective for reducing the levels of microorganisms in recreational water systems.

In another aspect, the present invention is directed to a composition for reducing the levels of microorganisms in recreational water systems, comprising: (1) 25-50 wt % of dibromonitrilopropionamide (DBNPA); (2) 25-50 wt % of didecyldimethylammonium chloride (DDAC); and (3) 0.1-50 wt % of a compound capable of in situ activation to form an oxidizing agent selected from the group consisting of sodium persulfate, potassium persulfate, ammonium persulfate, and combinations thereof; wherein all weight percents are based on the total weight of the composition, and the composition is effective for reducing the levels of microorganisms in recreational water systems.

In another aspect, the present invention is directed to a method of controlling the growth of microorganisms in recreational water systems, comprising the step of providing a composition comprising: (1) a biocidal effective amount of dibromonitrilopropionamide (DBNPA); and (2) optionally, a biocidal effective amount of an algaecide selected from the group consisting of didecyldimethylammonium chloride (DDAC), zinc, and copper; adding the composition to a recreational water system, adding to the recreational water system a compound capable of in situ activation to form an oxidizing agent; wherein the amount of the composition added to the water system is sufficient to provide a final DBNPA concentration of from 0.05 to 100 ppm.

These and other aspects will become apparent upon reading the following description of the invention.

### DETAILED DESCRIPTION OF THE INVENTION

It has been unexpectedly discovered that a biocide composition comprising 2,2-dibromo-3-nitrolopropionamide (DBNPA) and optionally didecyldimethylammonium chloride (DDAC) displays unexpected stability in swimming pool and spa applications, and is effective at sanitizing bodies of recreational water, such as pools, spas, and hot tubs. It was observed that significant residual levels of DBNPA and optionally DDAC were maintained in swimming pool and spa water under high water temperature, maximum sunlight, neutral to slightly alkaline pH, and presence of bather load through daily additions of liquid or daily delivery from tablets. The resulting residual levels of DBNPA and DDAC proved efficacious at preventing the establishment of common bacteria, fungi, and algae in the recirculating water system, or in a visible biofilm. Through regular applications of the composition of the invention, it has been demonstrated that microorganisms and biofilm can be remediated and growth significantly reduced so as not to compromise the circulation and filtration systems, or negatively impact water clarity. In the case where the biofilm problem has become manifest, the treatment method of the invention can restore plumbing, filter operation and water clarity by cleaning a significant amount of the biofilm from the circulation and filtration systems.

As defined herein, the term “effective amount” refers to an amount that achieves an effective result, and preferably reduces microorganisms by at least 50%.

The primary component of the composition of the invention is dibromonitrilopropionamide (DBNPA). The amount of DBNPA in the composition of the invention is any amount that results in a biocidal effect when added to a recirculating water system. In more specific embodiments, the amount of DBNPA in the composition ranges from 0.1% to 40% by weight as liquid or 1% to 99% by weight as solid (in granular or compacted forms). Preferably 3% to 30% by weight as liquid or 10% to 75% by weight as a solid, more preferably 5 to 25% by weight as liquid or 15% to 60% by weight as a solid, most preferably 10 to 20% by weight as a liquid and 25% to 50% by weight as a solid, all weight percents being based on the total weight of the composition.

To result in a biocidal effect, the biocidal effective amount of DBNPA in the composition preferably results in a final biocidal concentration in water of between about 0.05 and about 100 ppm, more preferably between about 0.1 and 50 ppm, and most preferably between about 0.25 and 25 ppm. One particularly useful concentration of DBNPA in water is about 2 ppm.

An optional ingredient in the composition of the invention is an algaecide selected from the group consisting of didecyldimethylammonium chloride (DDAC), zinc, or copper, where the zinc and copper are in the form of water soluble zinc or copper salts, such as copper sulfate, copper chloride, zinc sulfate, zinc chloride, and the like. The amount of algaecide that may be included in the composition of the invention ranges from 1% to 75% by weight as liquid or 5% to 75% by weight as solid (in granular or compacted forms). Preferably 5% to 70% by weight as liquid or 10% to 70% by weight as a solid, more preferably 10 to 60% by weight as liquid or 15% to 60% by weight as a solid, most preferably 20 to 50% by weight as a liquid and 25% to 50% by weight as a solid. Additionally, the most preferred concentration ratio of DBNPA: algaecide (as measured in PPM in the treated water) is 0.15:1.0 to 24:50. In use, the preferred amount of algaecide in the composition of the invention preferably results in a final concentration in water of algaecide of between about 0.2 and about 20 ppm, more preferably between about 0.5 and 10 ppm, and most preferably between about 1.0 and 5.0 ppm.

DBNPA and algaecide may be applied as an “initial dose” to “shock” the body of recreational water if it has a high concentration of microorganisms or biofilm. Following an initial shock, daily or weekly doses of this ingredient may be added to act as a maintenance/preventative step to prevent further growth of microorganisms and biofilm. Values of the initial dose, daily dose, and weekly dose of DBNPA and algaecide are shown below.

| | | | Weekly | |
|---|---|---|---|---|
| | Initial Dose | Daily Dose | Dose | |
| Product | (ppm) | (ppm) | (ppm) | Comment |
| DBNPA | 0.1-24.0 | 0.1-6.0 | 0.1-24 | Preferred Range |
| DBNPA | 0.5-12.0 | 0.5-4.0 | 6.0-20.0 | More Preferred |
| | | | | Range |
| DBNPA | 2.0-6.0 | 1.0-3.0 | 10.-18.0 | Most Preferred |
| | | | | Range |
| Algaecide | 0.2-20 | 0.05-24 | 0.2-5.0 | Preferred Range |
| Algaecide | 1.0-10 | 0.1-1.0 | 0.3-3.0 | More Preferred |
| | | | | Range |
| Algaecide | 2.0-4.0 | 0.15-0.3 | 0.4-2.4 | Most Preferred |
| | | | | Range |
| Persulfate | 2.0-50.0 | 1.0-12.0 | 2.0-50.0 | Preferred Range |
| Persulfate | 4.0-24.0 | 2.0-10.0 | 4.0-24.0 | More Preferred |
| | | | | Range |
| Persulfate | 8.0-20.0 | 4.0-8.0 | 8.0-20.0 | Most Preferred |
| | | | | Range |

The composition of the invention may optionally include a compound capable of in situ activation to form an oxidizing agent. “In situ activation” is defined herein as activation of a compound into an active oxidizing agent upon contact with recreational water. Examples of such compounds capable of in situ activation to form an oxidizing agent include sodium persulfates, potassium persulfates, and ammonium persulfates

In preferred embodiments relating to the composition of the invention, the optional compound capable of in situ oxidation may be sodium persulfate, potassium persulfate, ammonium persulfate, or a combination of these. The amount of compound capable of in situ activation preferably ranges from about 0.1 to about 50 wt %, more preferably from about 1.0 to about 35 wt %, and most preferably from about 2.0 to about 20 wt %, all weight percents being based on the total weight of the composition.

Like the DBNPA and algaecide above, the compound capable of in situ activation to form an oxidizing agent may be applied as an “initial dose” to “shock” the body of recreational water if it has a high concentration of microorganisms or biofilm. Following an initial shock, daily or weekly doses of this ingredient may be added to act as a maintenance/preventative step to prevent further growth of microorganisms and biofilm. Values of the initial dose, daily dose, and weekly dose are shown in the table above.

According to the invention, the composition may include DBNPA with or without algaecide and with or without a compound capable of in situ activation to form an oxidizing agent. Thus, in one embodiment, the composition of the invention may include DBNPA and an algaecide. In another embodiment, the composition of the invention may include DBNPA and a compound capable of in situ activation to form an oxidizing agent. In another embodiment, the composition of the invention may include DBNPA, an algaecide, and a compound capable of in situ activation to form an oxidizing agent.

According to the method of the invention, the above composition may be made without the in situ oxidation agent. In such circumstances, it is possible in the method of the invention to add an oxidizer separately. Any compound that generates active oxygen, hydroxyl radicals, ozone, chlorine dioxide, or free halogen (hypohalous acid) may be used for this purpose, for example, sodium persulfate, potassium persulfate, ammonium persulfate, sodium percarbonate, sodium perborate, calcium hypochlorite, sodium dichloroisocyanuric acid, trichloroisocyanuric acid, ammonium monopersulfate, or hydrogen peroxide as the oxidizing agent. When added separately, the in situ oxidizing agent may be applied as an “initial dose” to “shock” the body of recreational water if it has a high concentration of microorganisms or biofilm. Following an initial shock, daily or weekly doses of this ingredient may be added to act as a maintenance/preventative step to prevent further growth of microorganisms and biofilm. Values of the initial dose, daily dose, and weekly dose of hydrogen peroxide and potassium monopersulfate are shown below.

| | | | Weekly | |
|---|---|---|---|---|
| | Initial Dose | Daily Dose | Dose | |
| Product | (ppm) | (ppm) | (ppm) | Comment |
| Potassium | 1.0-50 | 0.5-6.0 | 4.0-50 | Preferred Range |
| Monopersulfate | | | | |
| Potassium | 6.0-36 | 0.75-3.0 | 6.0-36 | More Preferred |
| Monopersulfate | | | | Range |
| Potassium | 12-24 | 1.0-2.0 | 8.0-18 | Most Preferred |
| Monopersulfate | | | | Range |
| Hydrogen | 1.0-100 | 0.2-6.0 | 2.0-30 | Preferred Range |
| Peroxide | | | | |
| Hydrogen | 10-75 | 0.5-4.0 | 4.0-20 | More Preferred |
| Peroxide | | | | Range |
| Hydrogen | 20-56 | 0.75-2.0 | 7.0-14 | Most Preferred |
| Peroxide | | | | Range |

The compositions according to the present invention may also contain additives known in the water treatment art. These additives include but are not limited to pigments, dissolution rate modifiers, binders, lubricants, color-containing salts, biocides, buffers, chelating agents, other algaecides, fungicides, sequestering agents, clarifiers, enzymes, dyes, fragrances, surfactants, biodisperants, biopenetrants, sorbitan monostearate, sulfamic acid, tallowpropylamine diamine, cocopropylamine diamine, oleylpropylamine diamine, stearyldimethylbenzylammonium chloride, and combinations thereof. These additives may be pre-blended with any of the components of the composition, and are generally present in the composition of the invention in amounts ranging from 0.2 to 10 weight percent.

The composition and method of the present invention may be used in any recirculating water system where biofilm accumulates, for example swimming pools, spas, hot tubs, and decorative ponds. In use as a treatment for swimming pools, the composition of the invention is added to a swimming pool recirculating water system to achieve the above concentration ranges and demonstrates a synergistic effect between the ingredients. Since the composition of the invention provides that the compound capable of in situ activation to form an oxidizing agent is an optional component, a user may administer the composition without the compound capable of in situ activation. The user may add this ingredient separately at a later time as described above.

The routine application (preventative application) of a daily additions of DBNPA, algaecide, and optional compound capable of in situ activation to form an oxidizing agent, has shown a synergistic effect at preventing the establishment of common swimming pool bacteria and fungi in the water or in a visible biofilm that use of biocide alone could not achieve. Remedial treatments that include additions of DBNPA also show an effect at remediating established populations of common swimming pool bacteria and fungi in the water and plumbing and filter that the use of either biocide alone could not achieve.

The following examples are meant to illustrate, but in no way limit the present invention.

### EXAMPLES

### Example 1

The purpose of this experiment is to evaluate the robustness or the ability of single or combinations of biocides (plus additionally any other permutations of oxidizers and/or adjuvants) to prevent inoculated microorganisms from establishing colonies in the water and sand filter media, thus preventing bio-fouling of the system.

800 ml of synthetic swimming pool water (water containing calcium chloride dihydrate and sodium hydrogen carbonate solution at neutral pH) was pumped through a body of swimming pool filter sand by means of a peristaltic pump. The water temperature of each system was maintained in the range of 80-90° F. The biocide 2,2-dibromo-3-nitrilopropionamide (DBNPA) was added daily at a concentration of 2 ppm. When required, oxidizer hydrogen peroxide was added at a concentration of 27.5 ppm at the start of experiment, and 7 ppm weekly thereafter, and the loss of hydrogen peroxide in each system is monitored by calorimetric assay. In additional samples, potassium monopersulfate was provided at an initial dosage of 12 ppm, and then added weekly at a concentration of 12 ppm. To some samples, an additional algaecide (e.g., didecyldimethylammonium chloride (DDAC)) was added at an initial dosage concentration of 3 ppm and then 1 ppm weekly thereafter.

The synthetic swimming pool water samples dosed with combinations of DBNPA, hydrogen peroxide, potassium monopersulfate and/or DDAC were compared to the performance of the commercially available Baquacil® swimming pool chemical treatment system (available commercially from Arch Chemicals, Inc., Norwalk, Conn.). Baquacil is maintained at 6 ppm active ingredient (polyhexamethylene biguanide; PHMB), by addition of daily or weekly doses of the biocide. Hydrogen peroxide is added monthly at a concentration of 27.5 ppm.

The samples were challenged on a daily basis with eight species of bacteria and four species of fungi typically found in swimming pool water. These microorganisms include species of the fungi Paecilomyces and Trichoderma, and species of the bacteria Alcaligenes, Chryseobacterium and Sphingomonas. Each inoculation represents a total addition of 0.8×10⁶ microorganisms per model apparatus. In addition, 5 ml of synthetic bather load is added to the system on a daily basis, as a nutrient source for the microorganisms present in the system. The bather load consists of carbon, nitrogen and macro/micro nutrient sources such as urea, albumin, creatinine, lactic acid, uric acid, glucuronic acid, sodium chloride, sodium sulfate, ammonium chloride, sodium bicarbonate, potassium phosphate potassium sulfate.

The total number of viable bacteria and fungi present in each sample was determined weekly by conducting agar plate counts. Briefly, water samples were removed from each experimental vessel and serial dilutions were made (in 10⁻¹ steps, down to a 10⁻⁵ of the original sample). An aliquot of each dilution was spread onto dry Cystine Lactose Electrolyte Deficient agar plates (for enumeration of bacteria) and dry Sabaroud-Dextrose agar plates (for enumeration of fungi). Bacterial and fungal plates were incubated for 3 and 5 days respectively at 30° C. prior to enumeration of the number of viable organisms.

Model water turbidity was measured on a daily basis) using a Hach 2100P turbidimeter to measure water sample nephelometric turbidity units (NTUs). DBNPA measurements were conducted three times per week by colorimetric assay, by addition of 10 μl of 1N HCl, 1 ml of 2% (w/v) potassium iodide (PI) and 900 μl of water to 3 ml of the pool water sample. To enable DBNPA determination in the presence of hydrogen peroxide, catalase is added to water samples prior to assay, with shaking at room temperature for 40 minutes.

The concentration of DDAC and PHMB in systems was measured daily by calorimetric assay by reaction with 0.024% (w/v) Eosin Y and 10% (w/v) sodium acetate trihydrate solution and measurement of the resultant color formation at 540 nm. Beer's Law plots for DDAC and PHMB were constructed using solutions of known concentration, and the resultant plots were used to determine the concentration of DDAC or PHMB in water samples. Hydrogen peroxide concentration was determined by using Lovibond hydrogen peroxide low range test tablets in conjunction with a Lovibond PC22 photometer, operated according to the manufacturer's instructions.

The performance of the candidate treatment regimens was determined by the number of days the water clarity was maintained below 1.0 NTU (Nephelometric Turbidity Units). For the purposes of this experimentation, three determinations of water turbidity above 1 NTU during a seven day period was deemed to indicate system failure. In addition, the number of bacteria and fungi present in the water (determined as CFU or Colony Forming Units per ml of water) upon exceeding the turbidity threshold was determined, as it has been demonstrated that when the turbidity exceeds 1.0 NTU there are significant bacterial and fungal populations present in both water and filter sand. Also, a visible biofilm may be observed in the sand and tubing when turbidity exceeds 1.0 NTU.

The data in Table 1 below demonstrate the sanitizing performance of DBNPA at preventing the water from becoming turbid and controlling the bacteria and fungi that are added to the system. The data shown in Table 1 demonstrate that a system dosed with DBNPA alone maintained water clarity for a period of 59 days, which is comparable to the performance of the commercially available Baquacil system. A system dosed with a combination of DBNPA and hydrogen peroxide provides for identical performance as the DBNPA alone treatment. However, systems maintained on a DBNPA and potassium monopersulfate combination provided a synergistic effect for enhanced performance, as system water clarity was maintained over at least the entire 88 day testing period.

TABLE 1
Performance of DBNPA with and without Oxidizer at
Controlling Bacterial and Fungal Growth

| | | Bacterial Counts | Fungal Counts |
|---|---|---|---|
| | Number of Days | at Clarity of 1.0 | at Clarity |
| Biocide and | of Clarity below | NTU or above | of 1.0 NTU or |
| Concentration | 1.0 NTU | (CFU/ml) | above (CFU/ml) |
| DBNPA | 59 | 1.39 × 10⁵ | 5 × 10⁰ |
| DBNPA and | 59 | 4.95 × 10⁶ | 5 × 10⁰ |
| hydrogen | | | |
| peroxide | | | |
| DBNPA and | 88* | 3 × 10³ | 0 |
| potassium | | | |
| monopersulfate | | | |
| PHMB | 57 | 7.15 × 10⁷ | 0 |
| (Control) | | | |

*Test was terminated at day 88 without detectable loss of treatment performance

As shown in Table 2, addition of a DBNPA and DDAC combination provided for significantly enhanced synergistic sanitizing performance when added with in conjunction with an oxidizer. Water samples dosed with a DDAC and DBNPA combination, in conjunction with either hydrogen peroxide or potassium monopersulfate, both maintained water clarity for the testing period of 118 days. In the case of the system amended with hydrogen peroxide, a residual of this chemical was still detectable at the end of the testing period.

TABLE 2
Performance of the combination of DBNPA and DDAC, with
and without Oxidizer at Controlling Bacterial and Fungal Growth

| | Number of | | |
|---|---|---|---|
| | Days | Bacterial Counts | Fungal Counts |
| | of Clarity | at Clarity of 1.0 | at Clarity |
| Biocide and Oxidizer | below | NTU or above | of 1.0 NTU or |
| Concentration | 1.0 NTU | (CFU/ml) | above (CFU/ml) |
| DBNPA and DDAC | 22 | 7.1 × 10⁶ | 4.0 × 10² |
| DBNPA, DDAC and | 118^{#} | 0 | 0 |
| hydrogen peroxide | | | |
| DBNPA, DDAC and | 118^{#} | 0 | 0 |
| potassium | | | |
| monopersulfate | | | |

^{#}Tests were terminated at day 118 without detectable loss of treatment performance

The data presented above demonstrates the biocidal effect of DBNPA, as well as a synergistic, enhanced biocidal effect of a DBNPA and DDAC combination in the presence of an oxidizing agent.

While the invention has been described in combination with embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the appended claims. All patent applications, patents, and other publications cited herein are incorporated by reference in their entireties.

What is claimed is:

1. A composition for reducing the levels of microorganisms in recreational water systems, comprising:
(1) a biocidal effective amount of dibromonitrilopropionamide (DBNPA);
(2) optionally, a biocidal effective amount of an algaecide selected from the group consisting of didecyldimethylammonium chloride (DDAC), zinc, and copper; and
(3) optionally, a compound capable of in situ activation to form an oxidizing agent;
wherein said composition is effective for reducing the levels of microorganisms in recreational water systems.

2. The composition of claim 1, wherein the amount of said DBNPA ranges from 0.1% to 40% by weight as liquid or 1% to 99% by weight as solid, based on the total weight of said composition.

3. The composition of claim 1, wherein the amount of said DBNPA ranges from 3% to 30% by weight as liquid or 10% to 75% by weight as a solid, based on the total weight of said composition.

4. The composition of claim 1, wherein the amount of said DBNPA ranges from 5% to 25% by weight as liquid or 15% to 60% by weight as a solid, based on the total weight of said composition.

5. The composition of claim 1, wherein the amount of said DBNPA ranges from 10% to 20% by weight as a liquid and 25% to 50% by weight as a solid, based on the total weight of said composition.

6. The composition of claim 1, wherein said algaecide is DDAC.

7. The composition of claim 1, wherein the amount of said algaecide ranges from 1% to 75% by weight as liquid or 5% to 75% by weight as solid, based on the total weight of said composition.

8. The composition of claim 1, wherein the amount of said algaecide ranges from 5% to 70% by weight as liquid or 10% to 70% by weight as a solid, based on the total weight of said composition.

9. The composition of claim 1, wherein the amount of said algaecide ranges from 10% to 60% by weight as liquid or 15% to 60% by weight as a solid, based on the total weight of said composition.

10. The composition of claim 1, wherein the amount of said algaecide ranges from 20% to 50% by weight as a liquid and 25% to 50% by weight as a solid, based on the total weight of said composition.

11. The composition of claim 1, wherein the weight ratio of DBNPA to algaecide ranges from 0.15:1 to 24:50, as measured in ppm in water.

12. The composition of claim 1, wherein said compound capable of in situ activation to form an oxidizing agent is selected from the group consisting of sodium persulfate, potassium persulfate, ammonium persulfate, and combinations thereof.

13. The composition of claim 1, wherein the amount of said compound capable of in situ activation to form an oxidizing agent ranges from 0.1 wt % to 50 wt %, based on the total weight of said composition.

14. The composition of claim 1, wherein the amount of said compound capable of in situ activation to form an oxidizing agent ranges from 1.0 wt % to 35 wt %, based on the total weight of said composition.

15. The composition of claim 1, wherein the amount of said compound capable of in situ activation to form an oxidizing agent ranges from 2.0 wt % to 20 wt %, based on the total weight of said composition.

16. The composition of claim 1, further comprising one or more additional ingredients selected from the group consisting of pigments, dissolution rate modifiers, binders, lubricants, color-containing salts, biocides, buffers, chelating agents, other algaecides, fungicides, sequestering agents, clarifiers, enzymes, dyes, fragrances, surfactants, biodisperants, biopenetrants, sorbitan monostearate, sulfamic acid, tallowpropylamine diamine, cocopropylamine diamine, oleylpropylamine diamine, stearyldimethylbenzylammonium chloride, and combinations thereof.

17. The composition of claim 1, wherein said compound capable of in situ activation to form an oxidizing agent is sodium persulfate.

18. The composition of claim 1, where component 2 is present, and component 3 is not present.

19. The composition of claim 1, where component 3 is present, and component 2 is not present.

20. A composition for reducing the levels of microorganisms in recreational water systems, comprising:
(1) 25-50 wt % of dibromonitrilopropionamide (DBNPA);
(2) 25-50 wt % of didecyldimethylammonium chloride (DDAC); and
(3) 0.1-50 wt % of a compound capable of in situ activation to form an oxidizing agent selected from the group consisting of sodium persulfate, potassium persulfate, ammonium persulfate, and combinations thereof;
wherein all weight percents are based on the total weight of said composition, and said composition is effective for reducing the levels of microorganisms in recreational water systems.

21. A method of controlling the growth of microorganisms in recreational water systems, comprising the step of
providing a composition comprising:
(1) a biocidal effective amount of dibromonitrilopropionamide (DBNPA); and
(2) optionally, a biocidal effective amount of an algaecide selected from the group consisting of didecyldimethylammonium chloride (DDAC), zinc, and copper;
adding said composition to a recreational water system,
adding to said recreational water system a compound capable of in situ activation to form an oxidizing agent;
wherein the amount of said composition added to said water system is sufficient to provide a final DBNPA concentration of from 0.05 to 100 ppm.

22. The method of claim 21, wherein said composition provides a final DBNPA concentration in water of from 0.01 to 50 ppm.

23. The method of claim 21, wherein said composition provides a final DBNPA concentration in water of from 0.25 to 25 ppm.

24. The method of claim 21, wherein said composition provides for a final algaecide concentration in water of from 0.2 to 20 ppm.

25. The method of claim 21, wherein said composition provides a final algaecide concentration in water of from 0.5 to 10.0 ppm.

26. The method of claim 21, wherein said composition provides a final algaecide concentration in water of from 1.0 to 5.0 ppm.

27. The method of claim 21, wherein said composition provides a final oxidizer concentration in water of from 0.2 to 100 ppm.

28. The method of claim 21, wherein said composition provides for a final oxidizer concentration in water of from 0.5 to 75 ppm.

29. The method of claim 21, wherein said composition provides for a final oxidizer concentration in water of from 0.75 to 56 ppm.

30. The method of claim 21, wherein said compound capable of in situ activation to form an oxidizing agent is selected from the group consisting of sodium persulfate, potassium persulfate, ammonium persulfate, sodium percarbonate, sodium perborate, calcium hypochlorite, sodium dichloroisocyanuric acid, trichloroisocyanuric acid, ammonium monopersulfate, hydrogen peroxide, and combinations thereof.
